(19) United States
(12) Patent Application Publication
(10) Pub. No.: US 2007/0004745 A1
(43) Pub. Date: Jan. 4, 2007

(54) Methods of treating benign prostatic hyperplasia or lower urinary tract symptoms by using PDE 5 inhibitors

(75) Inventors: Cecil Pickett, Far Hills, NJ (US); Cynthia Cuffie-Jackson, Far Hills, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering-Plough Corporation

(21) Appl. No.: 11/387,280

(22) Filed: Mar. 23, 2006

Related U.S. Application Data
(60) Provisional application No. 60/665,348, filed on Mar. 25, 2005.

(51) Int. Cl. A61K 31/522; A61K 31/517

(52) U.S. Cl. 514/252.17; 514/263.24; 514/263.36

(57) ABSTRACT

The use of PDE 5 inhibitors in methods for the treatment of benign prostatic hyperplasia or lower urinary tract symptoms and other physiological disorders, as a monotherapy and in combination with other active agents is disclosed. For example, a representative compound useful in the methods of the invention is:

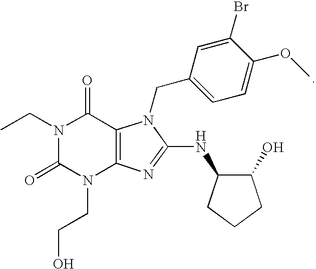

This application claims the benefit of U.S. provisional application No. 60/665,348, filed Mar. 25, 2005, which provisional application is incorporated by reference in its entirety.

### BACKGROUND OF THE INVENTION

1. Field of the Invention

The invention relates to the use of phosphodiesterase 5 inhibitors (“PDE 5”) in methods of preventing and/or treating benign prostatic hyperplasia (“BPH”) or lower urinary tract symptoms (“LUTS”).

2. Description of Related Art

BPH, a nonmalignant enlargement of the prostate, is the most common benign tumor in men. Approximately 50% of all men older than 65 years have some degree of BPH and a third of these men have clinical symptoms consistent with bladder outlet obstruction (Hieble, J. P. and Caine, M.,1986, “Etiology of benign prostatic hyperplasia and approaches to pharmacological management,” Fed. Proc. 45: 2601-2603). In the U.S., benign and malignant diseases of the prostate are responsible for more surgery than diseases of any other organ in men over the age of fifty.

The symptoms of the condition include, but are not limited to, increased difficulty in urination and sexual dysfunction. These symptoms are induced by enlargement, or hyperplasia, of the prostate gland. As the prostate increases in size, it impinges on free-flow of fluids through the male urethra. Concomitantly, the increased noradrenergic innervation of the enlarged prostate leads to an increased adrenergic tone of the bladder neck and urethra, further restricting the flow of urine through the urethra. These conditions can result in lower urinary tract symptoms, which may include increased frequency of urination, nocturia, a weak urine stream, hesitancy or delay in starting the urine flow and incomplete bladder emptying, hypertrophy of bladder smooth muscle, a decompensated bladder, an increased incidence of urinary tract infection, urinary stone formation and renal failure.

There are two components of BPH, a static component and a dynamic component. The static component is due to enlargement of the prostate gland, which may result in compression of the urethra and obstruction of the flow of urine from the bladder. The dynamic component is due to increased smooth muscle tone of the bladder neck and of the prostate itself (which interferes with emptying of the bladder), and is regulated by α1 adrenergic receptors (α1-ARs). The medical treatments available for BPH address these components to varying degrees, and the therapeutic choices are expanding.

Standard BPH treatment options include the following:

Watchful waiting: A strategy of management in which the patient is monitored but receives no active treatment.

Alpha blocker therapy: Treatment using alpha-1-adrenergic receptor blockers that inhibit contraction of prostatic smooth muscle.

Finasteride therapy: Treatment using finasteride (Proscar®), an enzyme inhibitor that lowers prostatic androgen levels and can result in some decrease of prostate size.

Transurethral incision of the prostate (TUIP): An endoscopic surgical procedure in which patients with smaller prostates (<30 g) have an instrument inserted through the urethra to make one or two cuts in the prostate and reduce the constriction on the urethra.

Transurethral resection of the prostate (TURP): Surgical removal of the prostates inner portion by endoscopic approach through the urethra. This is the most common active treatment.

Open prostatectomy: Surgical removal of the prostate via an incision in the lower abdomen. It usually requires a longer hospital stay.

Laser prostatectomy: Energy from directed neodynium yttrium aluminum garnet lasers is used to destroy prostate tissue. Initially bare laser fibers were used, with fairly disappointing results, but later technology advances enabled right angled fibers to direct the laser energy more directly at the tissue. The lasers are directed by ultrasound or direct cystoscopy.

Hyperthermia: Microwaves are used to locally heat the prostate tissue and destroy it. A number of technologies have been used to deliver microwaves transrectally or transurethrally.

Prostatic stents: Metal devices are placed in the prostatic urethra to expand the urethra and make urine flow easier.

Balloon dilation: A catheter with a balloon at the end is inserted through the urethra and into the prostatic urethra. The balloon is then inflated to stretch the urethra where narrowed by the prostate.

Surgical treatment options address the static component of BPH. TURP is the gold standard treatment for patients with BPH and approximately 320,000 TURPs were performed in the U.S. in 1990 at an estimated cost of $2.2 billion (Weis, K. A., Epstein R. S., Huse, D. M., Deverka, P. A. and Oster, G., 1993, “The costs of prostatectomy for benign prostatic hyperplasia,” Prostate 22: 325-334). Although an effective treatment for most men with symptomatic BPH, approximately 20-25% of patients do not have a satisfactory long-term outcome (Lepor, H. and Rigaud, G., 1990, “The efficacy of transurethral resection of the prostate in men with moderate symptoms of prostatism,” J. Urol. 143: 533-537). Complications include retrograde ejaculation (70-75% of patients), impotence (5-10%), postoperative urinary tract infection (5-10%), and some degree of urinary incontinence (2-4%) (Mebust, W. K., Holtgrewe, H. L., Cockett, A. T. K., and Peters, P. C., 1989, “Transurethral prostatectomy: immediate and postoperative complication: a cooperative study of 13 participating institutions evaluating 3,885 patients,” J. Urol., 141: 243-247). Furthermore, the rate of reoperation is approximately 15-20% in men evaluated for 10 years or longer (Wennberg, J. E., Roos, N., Sola, L., Schori, A, and Jaffe, R., 1987, “Use of claims data systems to evaluate health care outcomes: mortality and reoperation following prostatectomy,” JAMA 257: 933-936).

Apart from surgical approaches, there are some drug therapies which address the static component of this condition. Finasteride is a competitive inhibitor of the enzyme 5a-reductase, which is responsible for the conversion of testosterone to dihydrotestosterone in the prostate gland (Gormley, G., Stoner, E., Bruskewitz, R. C., et al., 1992, “The effect of finasteride in men with benign prostatic hyperplasia,” N. Engl. J. Med. 327: 1185-1191). Dihydrotestosterone appears to be the major mitogen for prostate growth, and agents which inhibit 5a-reductase reduce the size of the prostate and improve urine flow through the prostatic urethra. Although finasteride is a potent 5a-reductase inhibitor and causes a marked decrease in serum and tissue concentrations of dihydrotestosterone, it is only moderately effective in treating symptomatic BPH (Oesterling, J. E., 1995, “Benign prostatic hyperplasia: Medical and minimally invasive treatment options,” N. Engl. J. Med. 332: 99-109). The effects of finasteride take 6-12 months to become evident and for many men the clinical improvement is minimal.

The dynamic component of BPH has been addressed by the use of adrenergic receptor blocking agents (α1-AR blockers, “alpha blockers”), which act by decreasing the smooth muscle tone within the prostate gland itself. A variety of α1-AR blockers including terazosin (brand name Hytrin®), prazosin (brand name Minizide®), doxazosin (brand name Cardura®), tamsulosin (brand name Flomax®) and alfuzosin (brand name Uroxatral®), have been investigated for the treatment of symptomatic bladder outlet obstruction due to BPH, with terazosin being the most extensively studied. Although the α1-AR blockers are well-tolerated, approximately 10-15% of patients develop a clinically adverse event (Lepor, H., 1995, “alpha.-Blockade for benign prostatic hyperplasia (BPH),” J. Clin. Endocrinol. Metab. 80: 750-753). The undesirable effects of all members of this class are similar, with postural hypotension being the most commonly experienced side effect (Lepor, H., Auerbach, S., Puras-Baez, A. et al., 1992, “A randomized, placebo-controlled multicenter study of the efficacy and safety of terazosin in the treatment of benign prostatic hyperplasia,” J. Urol. 148:1467-1474). In comparison to the 5a-reductase inhibitors, the α1-AR blocking agents have a more rapid onset of action. However, their therapeutic effect, as measured by improvement in the symptom score and the peak urinary flow rate, is moderate. (Oesterling, 1995). The use of α1-AR antagonists in the treatment of BPH is related to their ability to decrease the tone of prostatic smooth muscle, leading to relief of the obstructive symptoms.

Certain families of PDE 5 inhibitor compounds and their use in treating a variety of physiological conditions are described in a number of patents (e.g., U.S. Pat. Nos. 6,821,978, 5,409,934, 5,470,579, 5,939,419 and 5,393,755) and foreign publications (e.g., WO 93/23401, WO 92/05176, WO 92/05175, and WO 99/24433). U.S. Pat. No. 6,821,978which is incorporated by reference in its entirety, describes a number of particularly active xanthine PDE 5 inhibitor compounds.

The use of PDE 5 inhibitors for treating impotence has met with commercial success with the introduction of sildenafil citrate (Viagra®, Pfizer, Connecticut, United States), vardenafil (Levitra®, Bayer, Germany) and tadalafil (Cialis®, Lilly-ICOS, Washington and Indiana, United States). The chemistry and use of Viagra®, including its mechanism of action in treating erectile dysfunction, are taught in EP 0 702 555 B1.

As has been shown by the representative art cited above, certain xanthine/guanine PDE 5 inhibitors have been found to be useful for treating cardiovascular and pulmonary disorders, while some others have been found useful for treating impotence.

### SUMMARY OF THE INVENTION

In one embodiment, the present invention comprises a method of treating benign prostatic hyperplasia or lower urinary tract symptoms comprising administering to a patient in need of such treatment an effective amount of at least one PDE 5 inhibitor compound, or an enantiomer, stereoisomer, rotomer, tautomer or a pharmaceutically acceptable salt thereof.

In some embodiments, the at least one PDE 5 inhibitor compound is selected from the group consisting of Compound Nos. 10-199, as herein defined.

In other embodiments, the at least one PDE 5 inhibitor compound is selected from the group consisting of Compound Nos. 60-65, 67, 103-107, 114-124, 128, 142, 160-161, 168-170, 176-181, 183, 186-188, 190, 191, 197 and 198, as herein defined.

In still other embodiments, the at least one PDE 5 inhibitor compound is selected from the group consisting of Compound Nos. 107, 114, 116, 118, 119, 122, 178, 186, 188, 191, 197 and 198.

In still other embodiments, the at least one PDE 5 inhibitor compound is selected from the group consisting of sildenafil, tadalafil, and vardenafil.

In still other embodiments, the at least one PDE 5 inhibitor compound is selected from the group consisting of:

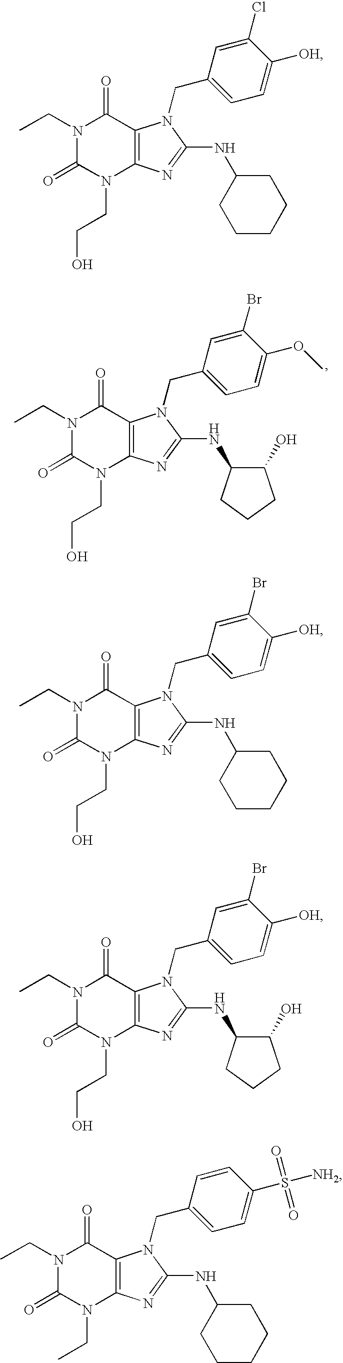

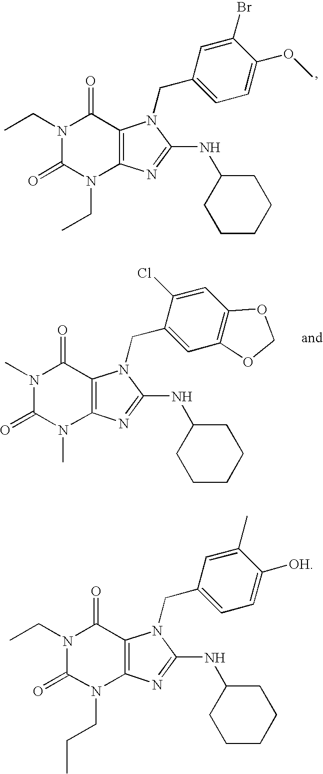

In yet another embodiment, the at least one PDE 5 inhibitor compound is:

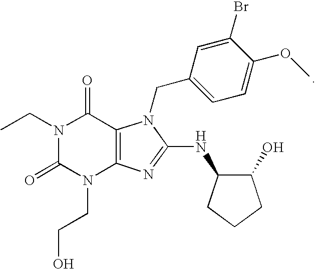

In still other embodiments, the at least one PDE 5 inhibitor compound is a compound of Formula (I), an enantiomer, stereoisomer, rotomer, tautomer or a pharmaceutically acceptable salt thereof:

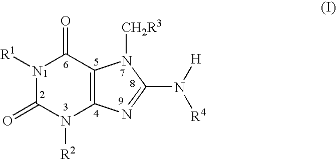

wherein:
(a) R¹ and R² are, independently of one another, each a C₁₋₁₅ alkyl group, branched or straight chain, unsubstituted or substituted with one or more substituents, a C₂₋₁₅ alkenyl group, branched or straight chain, unsubstituted or substituted with one or more substituents, a C₂₋₁₅ alkynyl group, branched or straight chain, unsubstituted or substituted with one or more substituents, or one of R¹ and R² is a hydrogen atom and the other one of R¹ and R² is defined the same as above;
(b) R³ is an aryl group, unsubstituted or substituted with one or more substituents, a heteroaryl group, unsubstituted or substituted with one or more substituents, or a heterocyclic group having 1 to 3 heteroatoms fused to a 5- or 6-membered aryl ring, unsubstituted or substituted with one or more substituents, with the proviso that R³ is not an aryl group substituted at its para position with a —Y-aryl group, where, Y is a carbon-carbon single bond, —C(O)—, —O—, —S—, —N(R²¹)—, —C(O)N(R²²)—, —N(R²²)C(O)—, —OCH₂—, —CH₂O—, —SCH₂—, —CH₂S—, —N(H)C(R²³)(R²⁴)—, —N(R²³)S(O₂)—, —S(O₂)N(R²³)—, —(R²³)(R²⁴)N(H)—, —CH═CH—, —CF═CF—, —CH═CF—, —CF═CH—, —CH₂CH₂—, —CF₂CF₂—, 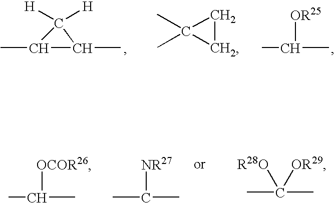
where,
   R²¹ is a hydrogen atom or a —CO(C₁₋₄ alkyl), C₁₋₆ alkyl, allyl, C₃₋₆ cycloalkyl, phenyl or benzyl group;
   R²² is a hydrogen atom or a C₁₋₆ alkyl group;
   R²³ is a hydrogen atom or a C₁₋₅ alkyl, aryl or —CH₂-aryl group;
   R²⁴ is a hydrogen atom or a C₁₋₄ alkyl group;
   R²⁵ is a hydrogen atom or a C₁₋₈ alkyl, C₁₋₈ perfluoroalkyl, C₃₋₆ cycloalkyl, phenyl or benzyl group;
   R²⁶ is a hydrogen atom or a C₁₋₆ alkyl, C₃₋₆ cycloalkyl, phenyl or benzyl group;
   R²⁷ is —NR²³R²⁴, —OR²⁴, —NHCONH₂, —NHCSNH₂, 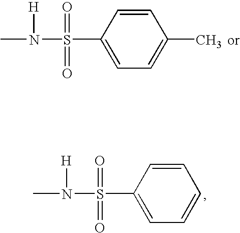
   and
   R²⁸ and R²⁹ are, independently of one another, each a C₁₋₄ alkyl group or, taken together with each other, a —(CH₂)_{q} group, where q is 2 or 3; and
(c) R⁴ is a C₃₋₁₅ cycloalkyl group, unsubstituted or substituted with one or more substituents, or a C₃₋₁₅ cycloalkenyl group, unsubstituted or substituted with one or more substituents;
wherein, the one or more substituents for all the groups are chemically-compatible and are, independently of one another, each an: alkyl, cycloalkyl, alkenyl, cycloalkenyl, alkynyl, arylalkyl, alkylaryl, aryl, heteroaryl, heterocycloalkyl, hydroxyalkyl, arylalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, alkoxy, hydroxy, halo, nitro, oximino, —COOR⁵⁰, —COR⁵⁰, —SO₀₋₂R⁵⁰, —SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, —CON(R⁵⁰R⁵¹), —OCOR⁵⁰, —OCON(R⁵⁰R⁵¹), —N(R⁵²)CO (R⁵⁰), —N(R⁵²)COOR⁵⁰ or —N(R⁵²)CON(R⁵⁰R⁵¹) group, where:
R⁵⁰, R⁵¹ and R⁵² are, independently of one another, each a hydrogen atom or a branched or straight-chain, optionally substituted, C₁₋₆ alkyl, C₃₋₆ cycloalkyl, C₄₋₆ heterocycloalkyl, heteroaryl or aryl group, or R⁵⁰ and R⁵¹ are joined together to form a carbocyclic or heterocyclic ring system, or R⁵⁰, R⁵¹ and R⁵² are, independently of one another, each: 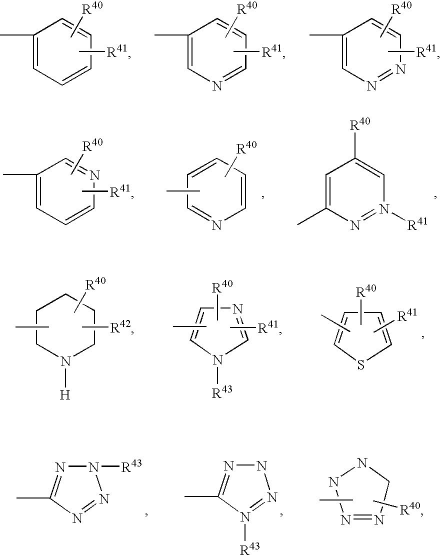
where,
   R⁴⁰ and R⁴¹ are, independently of one another, each a hydrogen atom or a branched or straight-chain, optionally substituted, alkyl, cycloalkyl, heterocycloalkyl, halo, aryl, imidazolylalkyl, indolylalkyl, heteroaryl, arylalkyl, arylalkoxy, heteroarylalkyl, heteroarylalkoxy, aminoalkyl, haloalkyl, mono-, di- or trihaloalkyl, mono-, di- or trihaloalkoxy, nitro, cyano, alkoxy, hydroxy, amino, phosphino, phosphate, alkylamino, dialkylamino, formyl, alkylthio, trialkylsilyl, alkylsulfonyl, arylsulfonyl, alkylsulfinyl, aminoalkyl, alkylaminoalkyl, dialkylaminoalkyl, hydroxyalkyl, morpholino, thioalkyl, alkylthioalkyl, carboxyalkyl, oximino, —COOR⁵⁰, —COR⁵⁰, —SO₀₋₂R⁵⁰, —SO₂NR⁵⁰R⁵¹, —NR⁵²SO₂R⁵⁰, —CON(R⁵⁰R⁵¹), —OCON(R⁵⁰R⁵¹), —N(R⁵²)CO(R⁵⁰), —N(R⁵²)COOR⁵⁰, —N(R⁵²)CON(R⁵⁰R⁵¹) or —OCONR⁵⁰ group, where, R⁵⁰, R⁵¹ and R⁵² are defined the same as above;
   R⁴² is a hydrogen atom or a branched or straight-chain, optionally substituted, alkyl, alkenyl, arylalkyl or acyl group; and
   R⁴³ is a hydrogen atom or a branched or straight-chain, optionally substituted, alkyl or aryl group;
   wherein, the optional substituents are defined the same as above for the one or more substituents.

In some embodiments, R¹ is a methyl or ethyl group, with or without the one or more substituents.

In some embodiments, R² is a methyl, ethyl, iso-butyl or hydroxyethyl group, with or without the one or more substituents.

In some embodiments, R³ is a phenyl group, with or without the one or more substituents.

In some embodiments, the phenyl group for R³ is substituted with at least one halogen atom.

In some embodiments, R⁴ is a cyclohexyl, hydroxycyclopentyl or tetrahydropyranyl group, with or without the one or more substituents.

In yet other embodiments, the PDE 5 inhibitor is selected from the group of compounds reflected in Tables I and II, infra.

In some embodiments the invention further comprises administering to the patient an effective amount of at least one active agent selected from the group consisting of finasteride, (α)1-AR blockers, prostanoids, α-adrenergic receptor, dopamine receptor agonists, melanocortin receptor agonists, endothelin receptor antagonists, endothelin converting enzyme inhibitors, angiotensin II receptor antagonists, angiotensin converting enzyme inhibitors, neutral metalloendopeptidase inhibitors, renin inhibitors, serotonin 5-HT_{2c} receptor agonists, nociceptin receptor agonists, rho kinase inhibitors, potassium channel modulators and inhibitors of multidrug resistance protein 5.

In some embodiments, the (α)1-AR blocker is selected from the group consisting of terazosin, prazosin, doxazosin, tamsulosin and alfuzosin.

In other embodiments, the invention further comprises administering to said patient at least one cardiovascular agent selected from the group consisting of thromboxane A2 biosynthesis, thromboxane antagonists, adenosine diphosphate (ADP) inhibitors, cyclooxygenase inhibitors, angiotensin antagonists, and endothelin (“ET_{A}”) antagonists. Non-limiting examples of ET_{A} antagonists include bosentan, atrasentan, ambrisentan, darusentan, sitaxsentan, ABT-627, TBC-3711, C1-1034, SPP-301, SB-234551, ZD-4054, BQ-123 and BE-18257B.

In some embodiments, the method further comprises treating said patient with a procedure selected from the group consisting of prostatic hyperthermia, prostatic stenting, and balloon dilation.

In some embodiments, the patients in the above methods do not suffer from LUTS prior to being treated with the PDE 5 inhibitor compound.

In some embodiments, the invention comprises a pharmaceutical composition for treating benign prostatic hyperplasia or lower urinary tract symptoms, said composition comprising an effective amount of a PDE 5 inhibitor compound and a pharmaceutically acceptable excipient.

In some embodiments of the composition, the PDE 5 inhibitor compound is:

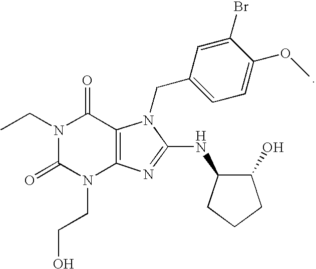

In some embodiments, the active agent or the cardiovascular agent is co-administered in a pharmaceutical dosage form that is distinct from that pharmaceutical dosage form comprising the PDE 5 inhibitor. In other embodiments, the PDE 5 inhibitor and the active agent or the cardiovascular agent are present in the same pharmaceutical dosage form.

A further understanding of the invention will be had from the following description of preferred embodiments.

### DEFINITIONS AND USAGE OF TERMS

The following definitions and terms are used herein or are otherwise known to a skilled artisan. Except where stated otherwise, the following definitions apply throughout the specification and claims. These definitions apply regardless of whether a term is used by itself or in combination with other terms, unless otherwise indicated.

The terms “excipient” and “pharmaceutically-acceptable excipient,” as used herein, include any physiologically inert, pharmacologically inactive material known to one skilled in the art, which is compatible with the physical and chemical characteristics of the particular active ingredient selected for use. Pharmaceutically-acceptable excipients include polymers, resins, plasticizers, fillers, binders, lubricants, glidants, disintegrates, solvents, co-solvents, buffer systems, surfactants, preservatives, sweetening agents, flavoring agents, pharmaceutical grade dyes or pigments, and viscosity agents.

The term “pharmaceutical composition,” as used herein, means a combination of at least one subject compound (e.g., PDE 5 inhibitor) and at least one pharmaceutically-acceptable excipient.

The term “pharmaceutically-acceptable salt,” as used herein, means a cationic salt formed at an acidic (e.g., carboxyl) group or an anionic salt formed at a basic (e.g., amino) group of the compound. Preferred cationic salts include the alkali-metal salts (e.g., sodium and potassium) and alkaline earth metal salts (e.g., magnesium and calcium). Preferred anionic salts include the halide (e.g., chloride), acetate and phosphate salts.

The phrase “effective amount,” as used herein, means an amount of a compound or composition which is sufficient to significantly and positively modify the symptoms and/or conditions to be treated (e.g., provide a positive clinical response). The phrase “safe and effective amount,” as used herein, means that an “effective amount” must also be safe, that is, an amount that is sufficient to provoke a positive response, yet is small enough to avoid serious side effects (at a reasonable benefit/risk ratio), within the scope of sound medical judgment. The effective amount of an active ingredient for use in a pharmaceutical composition will vary with the particular condition being treated, the severity of the condition, the duration of the treatment, the nature of concurrent therapy, the particular active ingredient being employed, the particular pharmaceutically-acceptable excipients utilized and like factors within the knowledge and expertise of the attending physician.

The phrase “administering [to a patient a safe and effective amount of the subject compound],” as used herein, refers to any mode of introducing any form (e.g., solid, liquid or gas) of a PDE 5 inhibitor compound in vivo to a patient (e.g., human or mammal). For example, introduction of the subject compound to a patient may be accomplished via oral ingestion (e.g., tablets, capsules, gels, solutions, etc.), adsorption, absorption (e.g., transmucosal sublingual or buccal administration), transdermal applications (e.g., topical applications via patches, lotions, etc.), suppositories, etc.

The term “oral dosage form,” as used herein, means any pharmaceutical composition intended to be systemically administered to an individual by delivering the composition to the gastrointestinal tract of an individual, via the mouth of the individual. For purposes of the invention, the delivered form can be a tablet (coated or non-coated), solution, suspension or capsule (coated or non-coated).

The term “injection,” as used herein, means any pharmaceutical composition intended to be systemically administered to a human or other mammal, via delivery of a solution or emulsion containing the active ingredient, by puncturing the skin of said individual, in order to deliver the solution or emulsion to the circulatory system of the individual either by intravenous, intramuscular, intraperitoneal or subcutaneous injection.

The terms “treating” and “treatment” are understood to encompass either amelioration of an existing or developing physical condition, or prophylactic prevention of the said physical condition.

The term “method of treating benign prostatic hyperplasia or lower urinary tract symptoms” is understood to encompass methods of treating benign prostatic hyperplasia in the presence or absence of lower urinary tract symptoms, and methods of treating lower urinary tract symptoms in the presence or absence of benign prostatic hyperplasia.

The term “patient” as used herein means mammal, including human.

The term “at least one” as used herein means one, two or three.

The term “PDE 5 inhibitor compound”, as used herein, means a compound that inhibits the PDE 5 receptor. Examples of PDE 5 inhibitor compounds include, but are not limited to, the compounds of Formula I and of Tables I and II from U.S. Pat. No. 6,821,978, sildenafil citrate (Viagra®), Pfizer, Connecticut, United States), vardenafil (Levitra®, Bayer, Germany) and tadalafil (Cialis®, Lilly-ICOS, Washington and Indiana, United States).

The definitions of any terms not defined herein but defined in U.S. Pat. No. 6,821,978 are incorporated herein by reference from U.S. Patent No. 6,821,978.

Unless otherwise indicated, all numbers used in the specification and claims expressing quantities of ingredients, reaction conditions, and so forth, are understood as being modified in all instances by the term “about.”

### DETAILED DESCRIPTION OF THE INVENTION

The present invention encompasses a method of medical management of benign prostatic hyperplasia and/or lower urinary tract symptoms in a male subject in need of such treatment by administering a therapeutically effective amount of at least one PDE 5 inhibitor compound, or a pharmaceutical composition thereof.

Examples of PDE 5 inhibitor compounds useful for treatment of BPH and/or LUTS include the xanthine derivative compounds described in U.S. Pat. No. 6,821,978, as represented by the following formula:

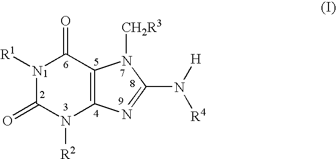

wherein the variables are as defined supra.

The following compounds, listed in Tables I and II of U.S. Pat. No. 6,821,978, are illustrative of these xanthine derivative compounds.

It is to be understood that any reference to Compound Numbers herein is a reference to the compound corresponding to the indicated number as found in either Table I or Table II. Thus, for example, reference to “Compound Nos. 10-199” is a reference to the compounds corresponding to compound nos 10-199 in Tables I and II.

TABLE I

| Compound | |
|---|---|
| No. | Structure |
| 10 | 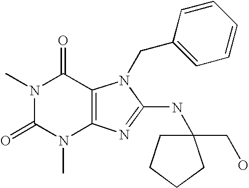 |
| 11 | 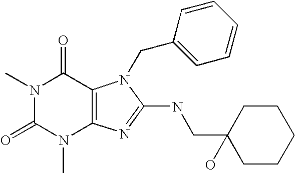 |
| 12 | 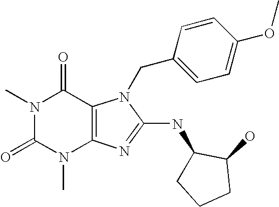 |
| 13 | 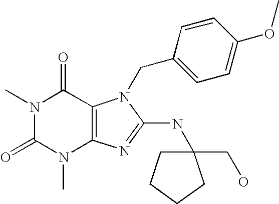 |
| 14 | 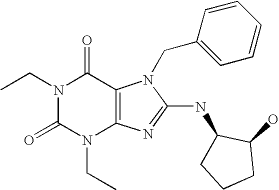 |
| 15 | 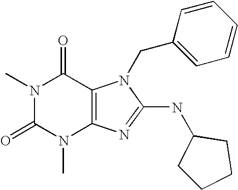 |
| 16 | 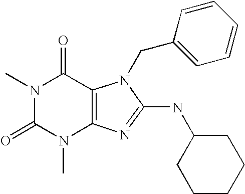 |
| 17 | 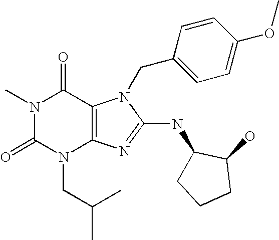 |
| 18 | 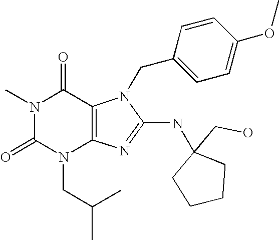 |
| 19 | 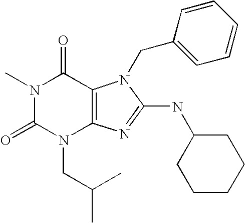 |
| 20 | 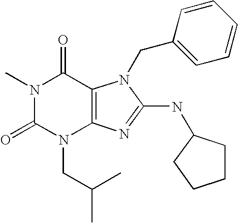 |
| 21 | 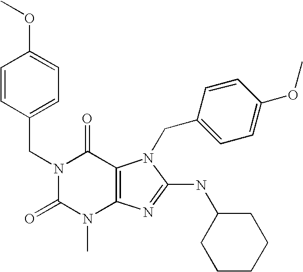 |
| 22 | 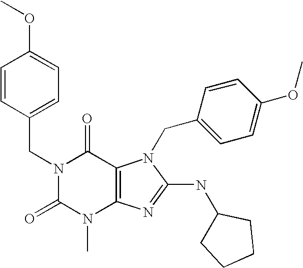 |
| 23 | 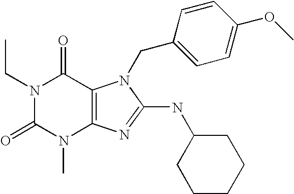 |
| 24 | 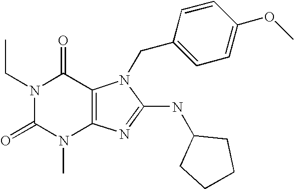 |
| 25 | 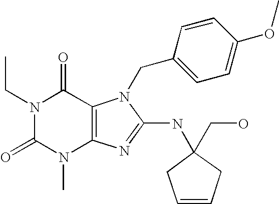 |
| 28 | 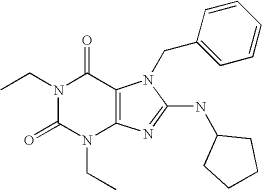 |
| 29 | 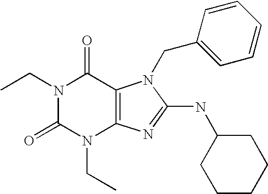 |
| 30 | 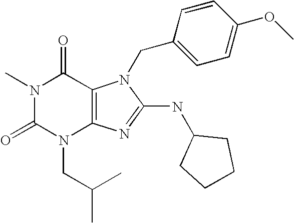 |
| 31 | 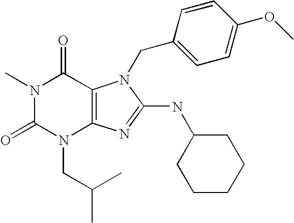 |
| 32 | 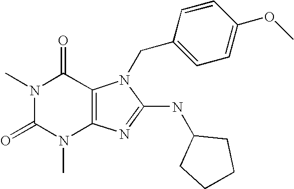 |
| 33 | 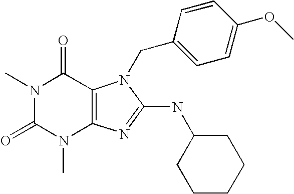 |
| 34 | 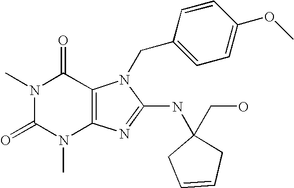 |
| 35 | 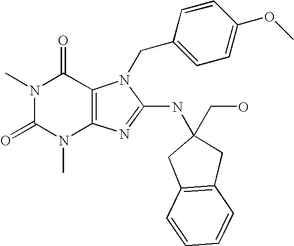 |
| 36 | 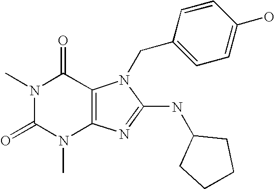 |
| 37 | 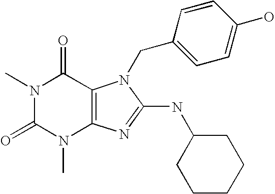 |
| 38 | 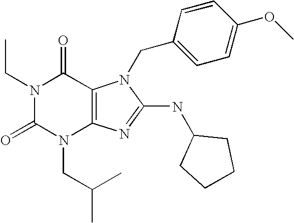 |
| 39 | 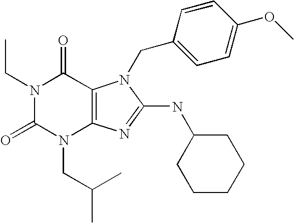 |
| 40 | 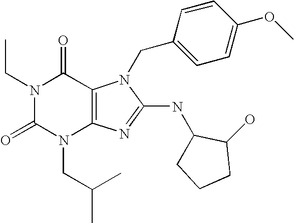 |
| 41 | 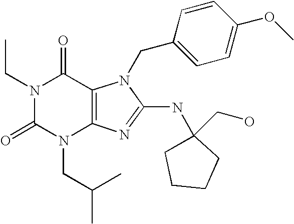 |
| 42 | 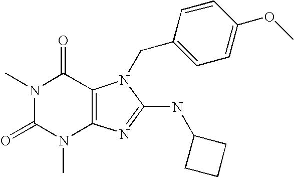 |
| 43 | 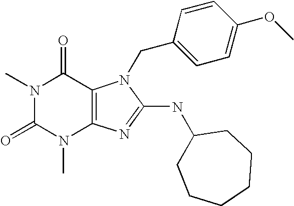 |
| 44 | 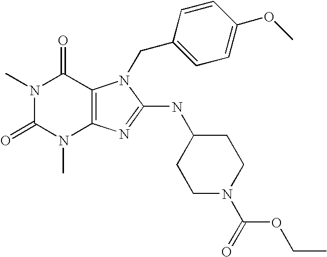 |
| 47 | 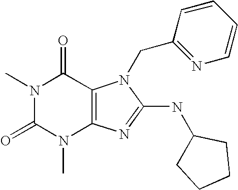 |
| 48 | 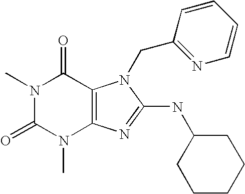 |
| 49 | 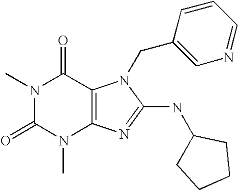 |
| 50 | 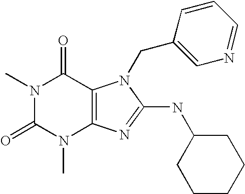 |
| 51 | 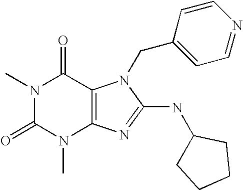 |
| 52 | 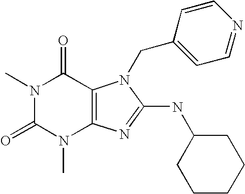 |
| 53 | 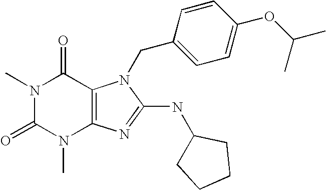 |
| 54 | 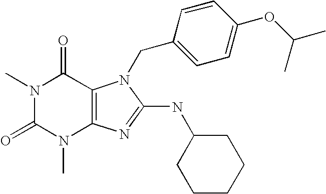 |
| 55 | 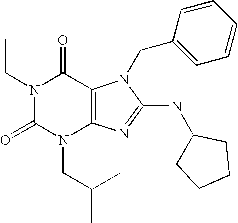 |
| 56 | 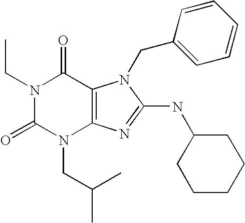 |
| 57 | 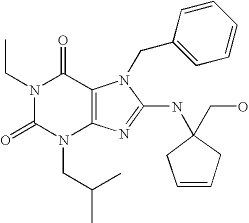 |
| 58 | 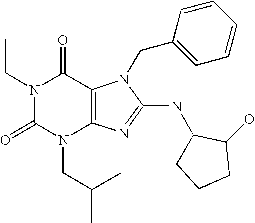 |
| 59 | 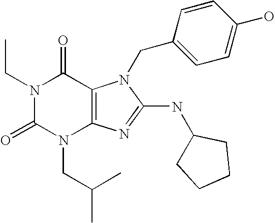 |
| 60 | 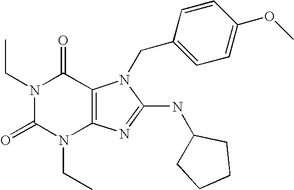 |
| 61 | 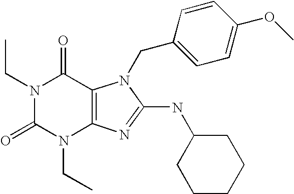 |
| 62 | 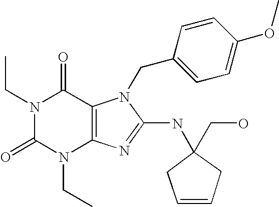 |
| 63 | 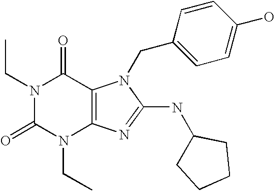 |
| 64 | 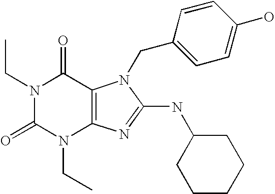 |
| 65 | 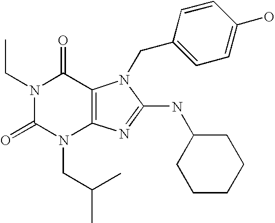 |
| 66 | 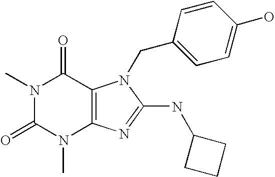 |
| 67 | 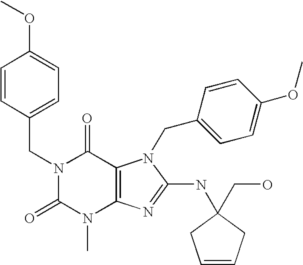 |
| 68 | 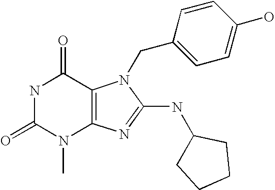 |
| 69 | 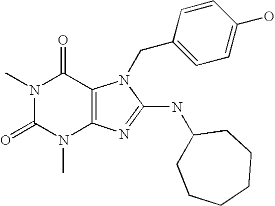 |
| 70 | 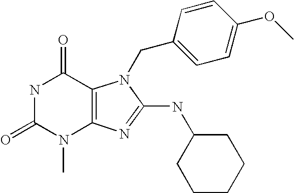 |
| 71 | 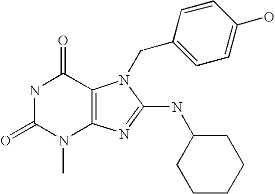 |
| 72 | 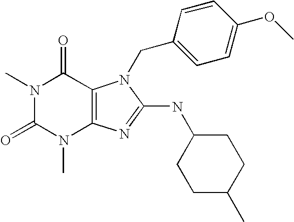 |
| 73 | 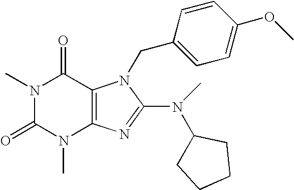 |
| 74 | 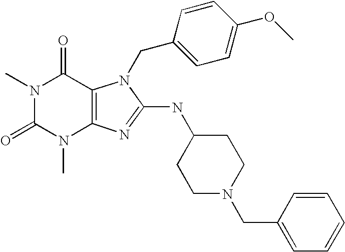 |
| 75 | 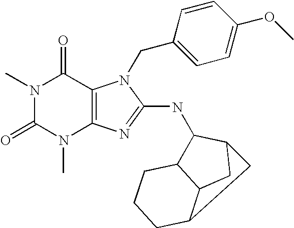 |
| 76 | 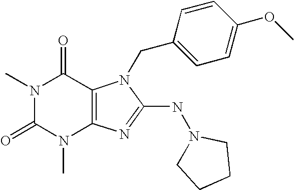 |
| 77 | 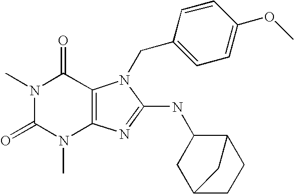 |
| 78 | 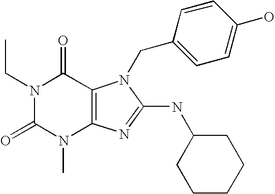 |
| 79 | 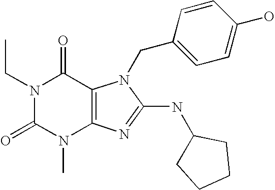 |
| 80 | 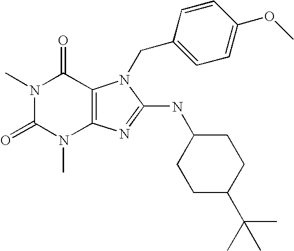 |
| 81 | 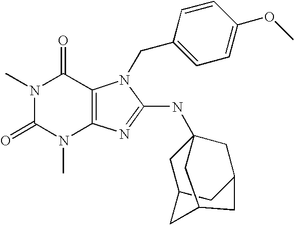 |
| 82 | 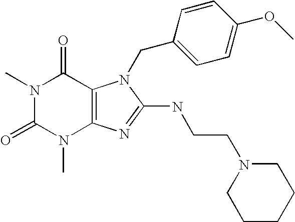 |
| 83 | 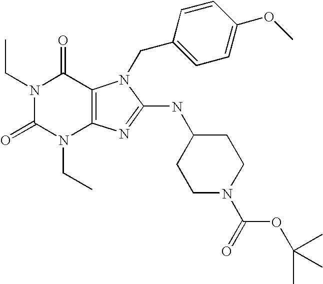 |
| 84 | 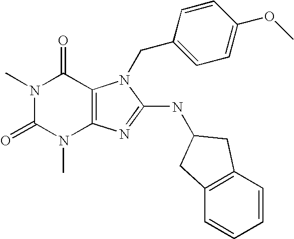 |
| 85 | 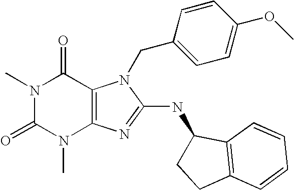 |
| 86 | 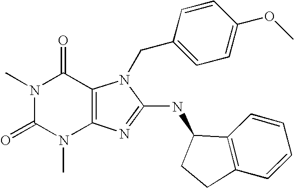 |
| 87 | 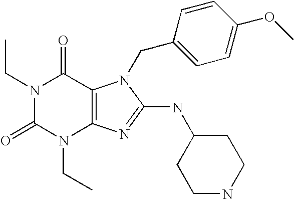 |
| 88 | 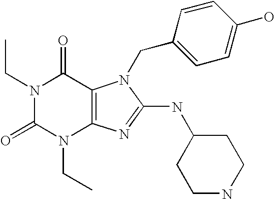 |
| 89 | 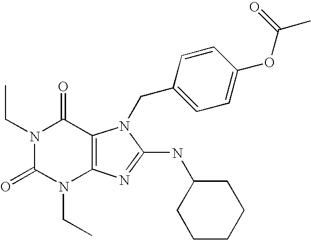 |
| 90 | 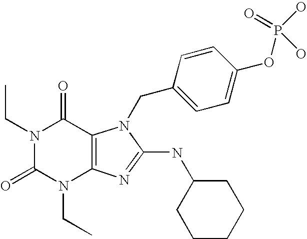 |
| 91 | 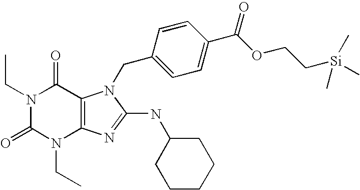 |
| 92 | 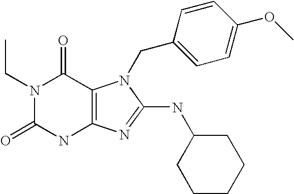 |
| 93 | 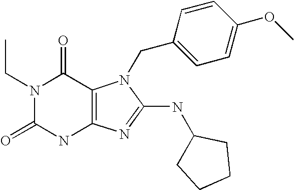 |
| 94 | 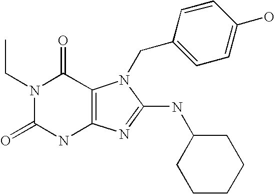 |
| 95 | 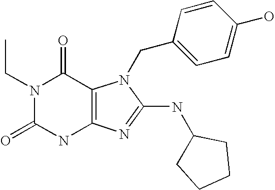 |
| 96 | 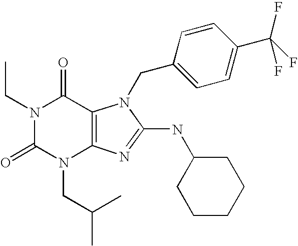 |
| 97 | 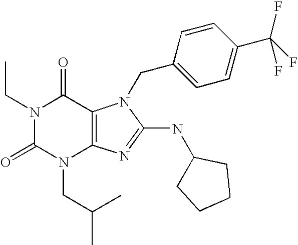 |
| 98 | 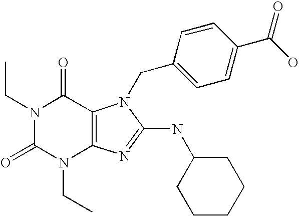 |
| 99 | 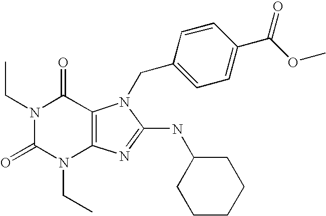 |
| 100 | 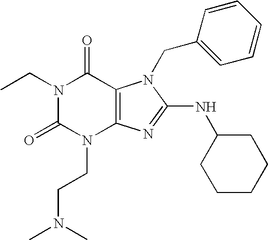 |
| 101 | 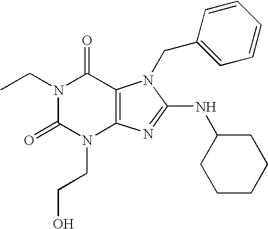 |
| 102 | 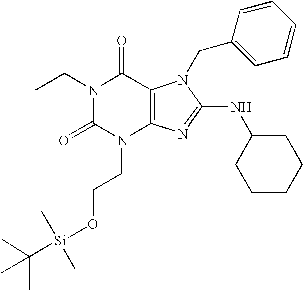 |
| 103 | 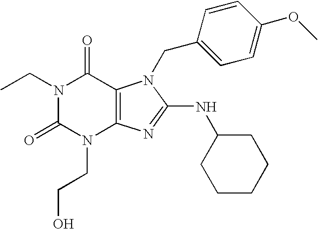 |
| 104 | 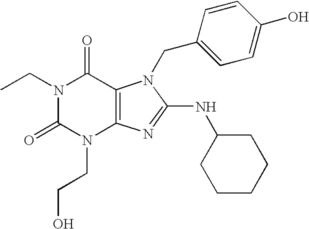 |
| 105 | 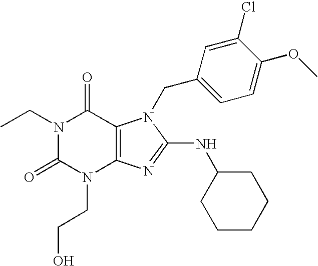 |
| 106 | 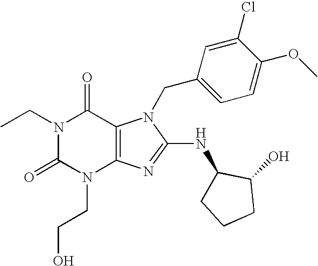 |
| 107 | 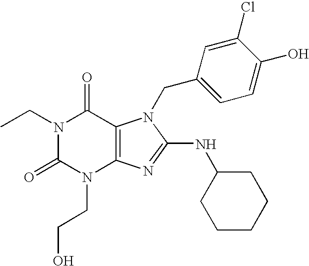 |
| 108 | 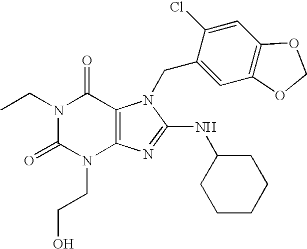 |
| 109 | 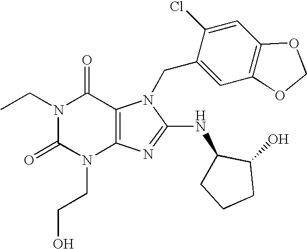 |
| 110 | 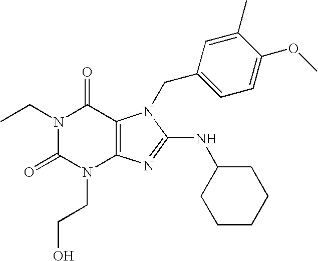 |
| 111 | 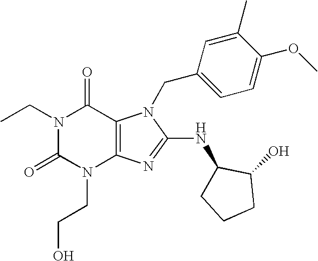 |
| 112 | 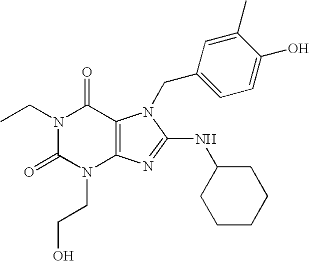 |
| 113 | 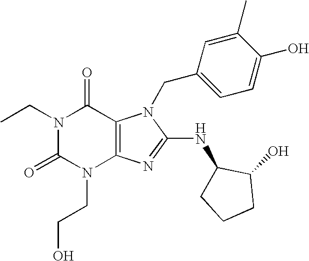 |
| 114 | 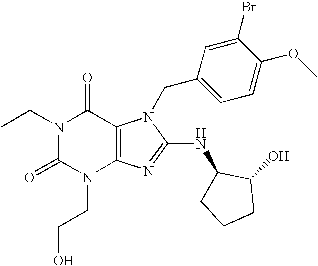 |
| 115 | 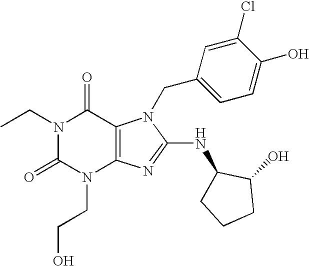 |
| 116 | 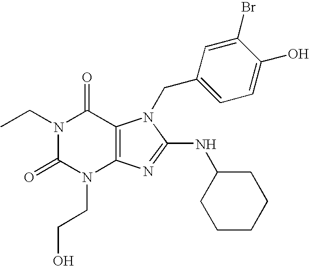 |
| 117 | 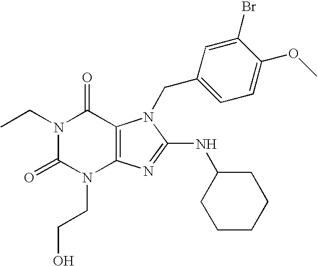 |
| 118 | 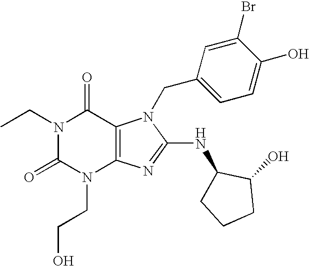 |
| 119 | 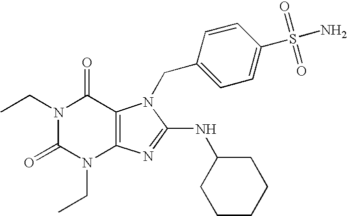 |
| 120 | 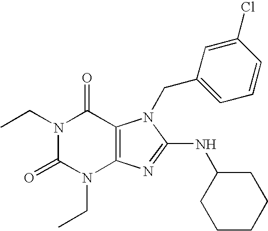 |
| 121 | 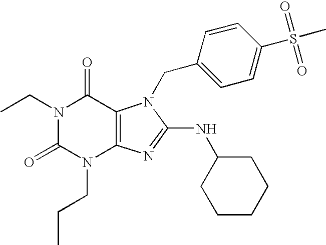 |
| 122 | 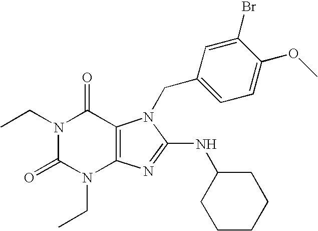 |
| 123 | 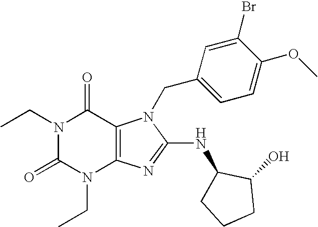 |
| 124 | 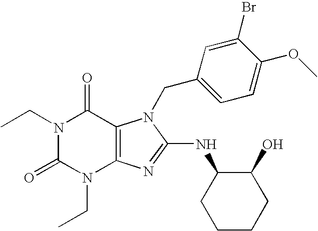 |
| 125 | 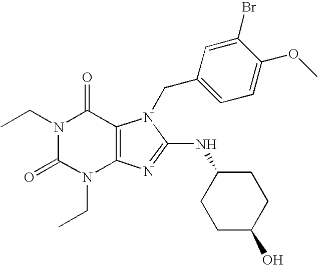 |
| 126 | 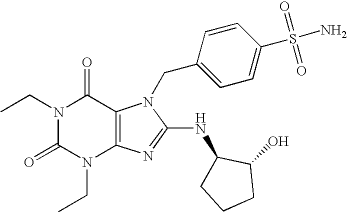 |
| 127 | 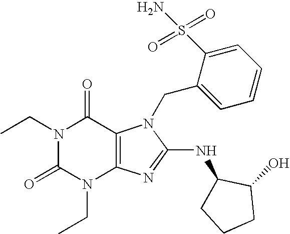 |
| 128 | 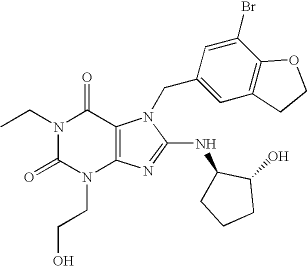 |
| 129 | 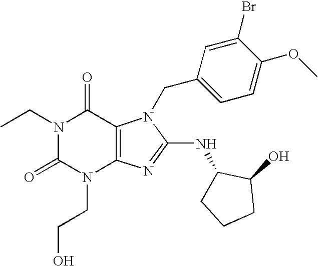 |
| 130 | no structure |
| 131 | 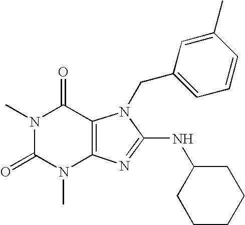 |
| 132 | 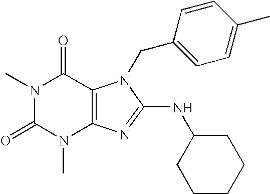 |
| 133 | 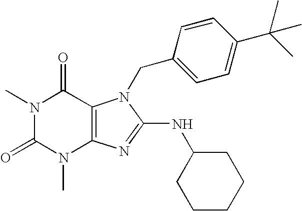 |
| 134 | 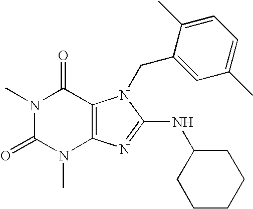 |
| 135 | 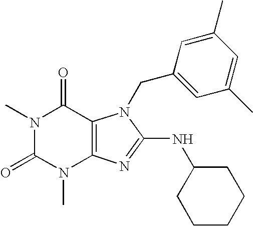 |
| 136 | 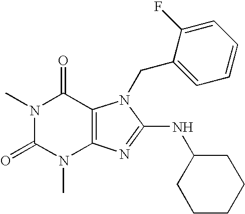 |
| 137 | 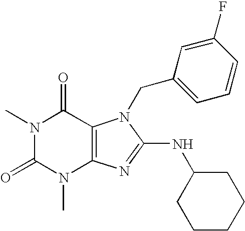 |
| 138 | 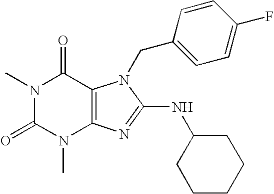 |
| 139 | 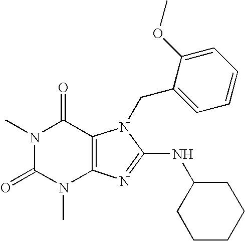 |
| 140 | 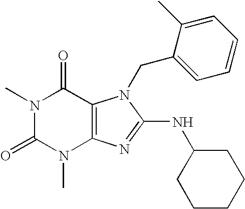 |
| 141 | 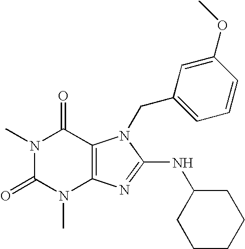 |
| 142 | 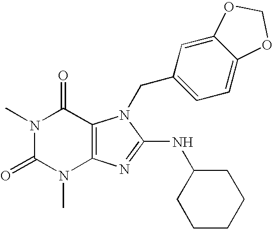 |
| 143 | 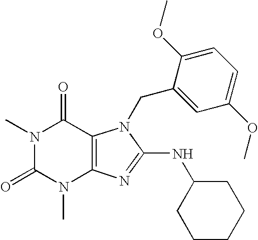 |
| 144 | 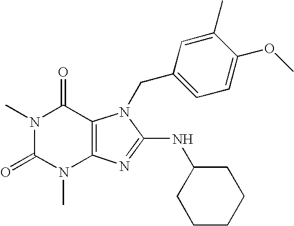 |
| 145 | 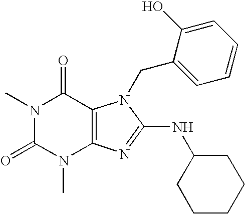 |
| 146 | 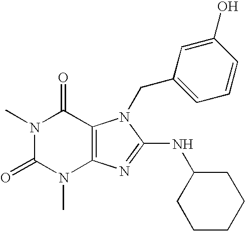 |
| 147 | 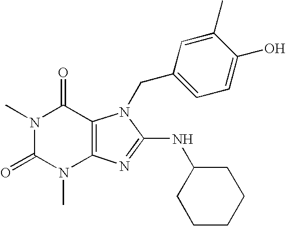 |
| 148 | 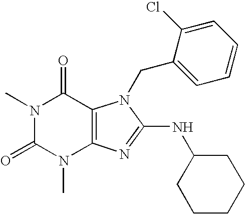 |
| 149 | 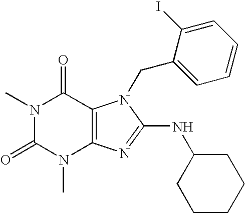 |
| 150 | 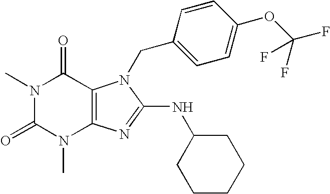 |
| 151 | 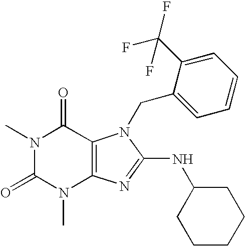 |
| 152 | 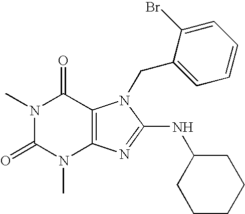 |
| 153 | 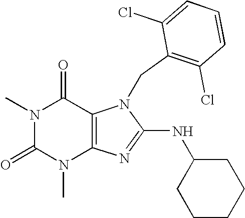 |
| 154 | 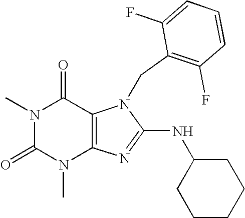 |
| 155 | 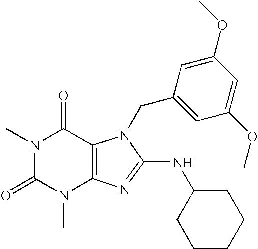 |
| 156 | 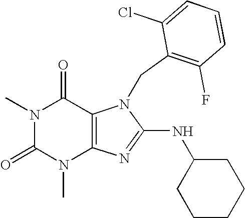 |
| 157 | 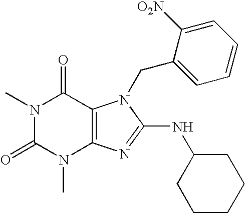 |
| 158 | 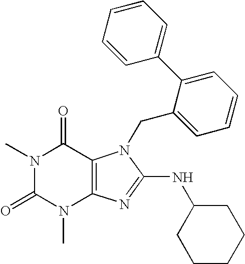 |
| 159 | 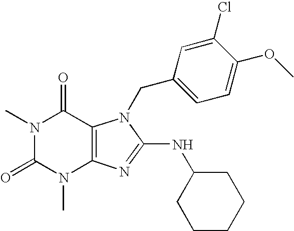 |
| 160 | 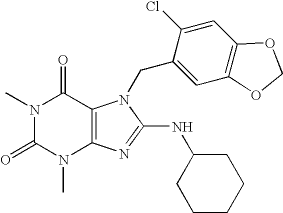 |
| 161 | 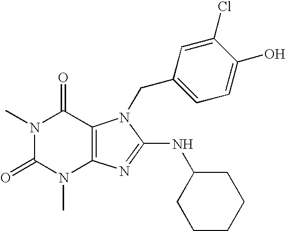 |
| 162 | 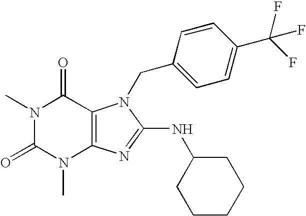 |
| 163 | 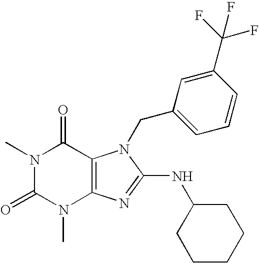 |
| 164 | 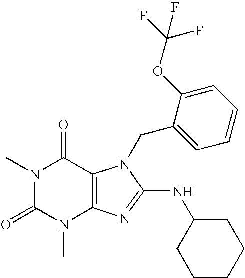 |
| 165 | 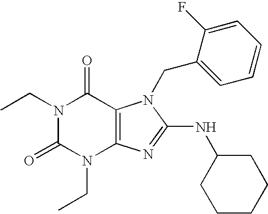 |
| 166 | 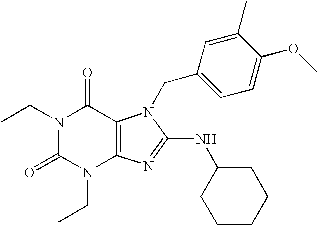 |
| 167 | 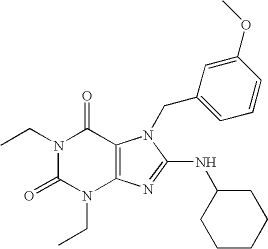 |
| 168 | 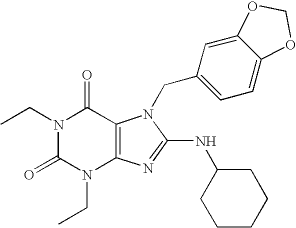 |
| 169 | 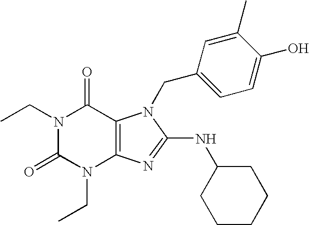 |
| 170 | 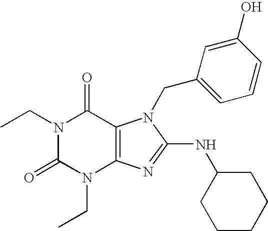 |
| 171 | 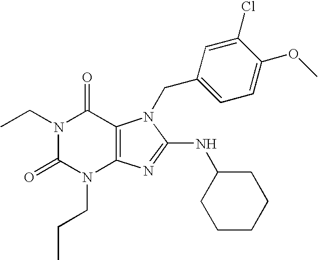 |
| 172 | 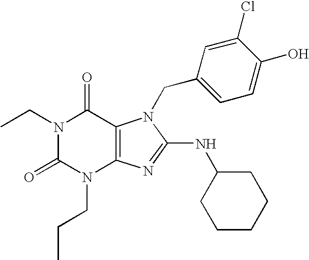 |
| 173 | 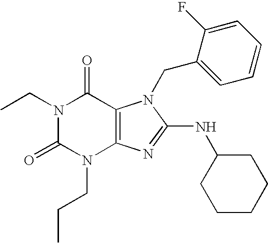 |
| 174 | 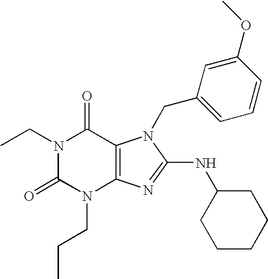 |
| 175 | 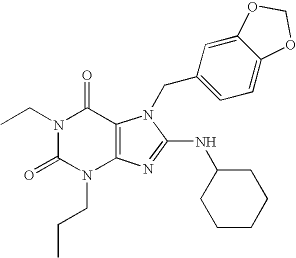 |
| 176 | 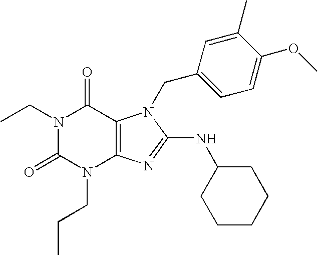 |
| 177 | 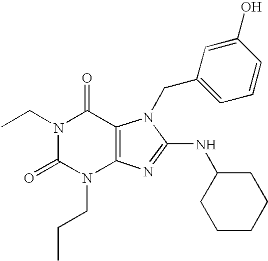 |
| 178 | 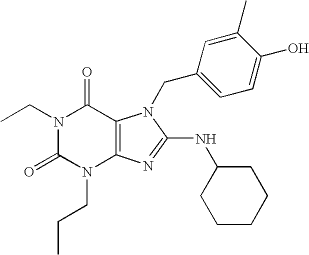 |
| 179 | 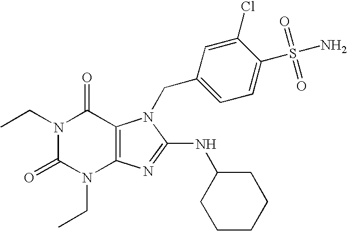 |
| 180 | 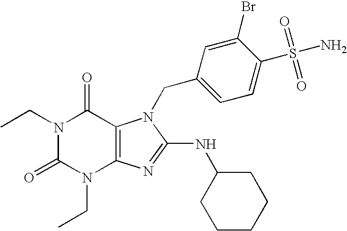 |
| 181 | 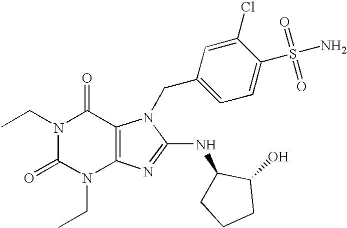 |
| 182 | 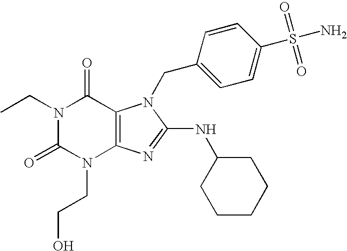 |
| 183 | 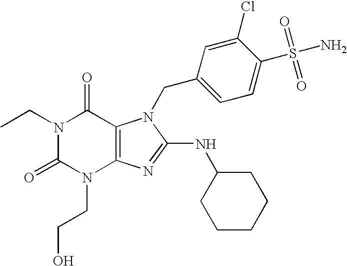 |
| 184 | 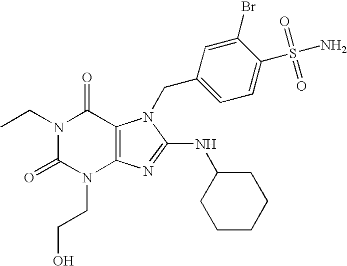 |
| 185 | 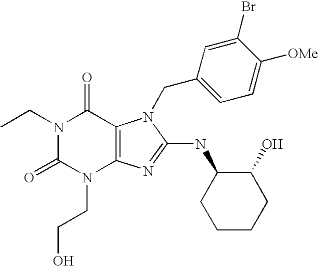 |
| 186 | 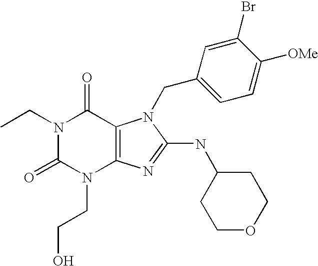 |
| 187 | 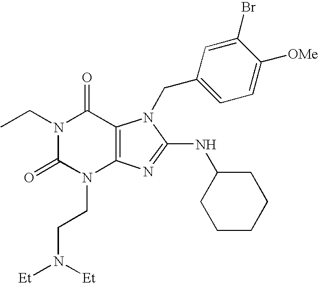 |
| 188 | 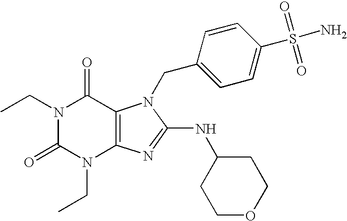 |
| 189 | 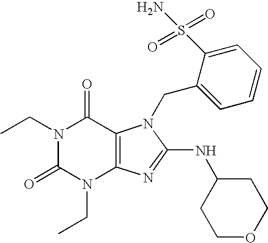 |
| 190 | 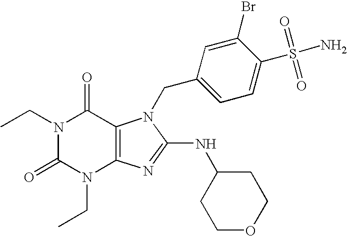 |
| 191 | 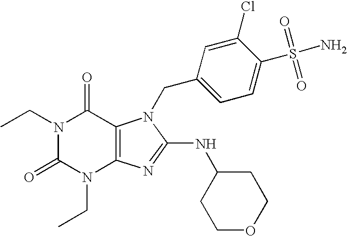 |
| 192 | 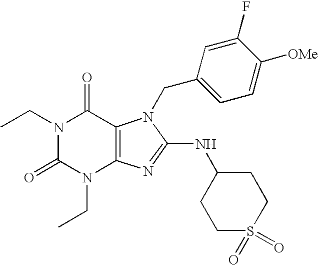 |
| 193 | 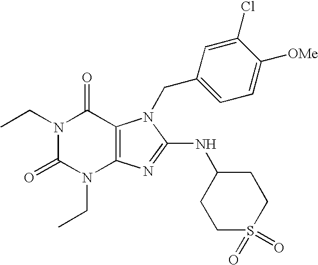 |
| 194 | 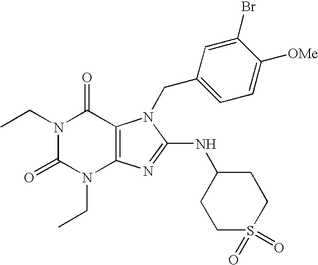 |
| 195 | 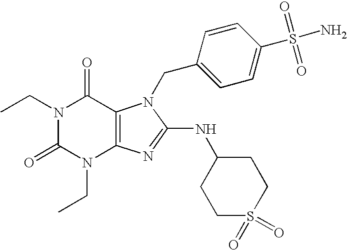 |
| 196 | 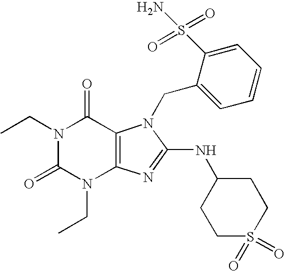 |
| 197 | 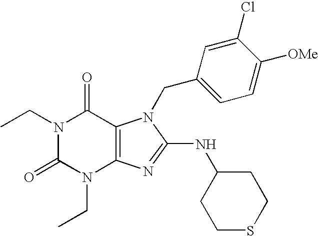 |
| 198 | 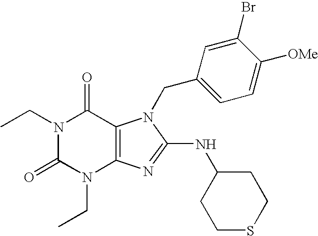 |
| 199 | 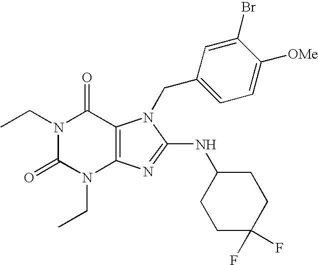 |

These compounds are useful for inhibiting PDE 5 enzymes. Their enzyme activities and enzyme selectivities can be evaluated in a number of 5 ways. In particular, enzyme activity can be measured by the PDE 5 IC₅₀ value, which is the concentration (in nM) of the compound required to provide 50% inhibition of PDE 5. The lower the value of IC₅₀, the more active is the compound. Measurements on the compounds in Tables I and II gave the following data (all numbers are modified by the word “about”):
A. all compounds (nos. 10-199) had a PDE 5 IC₅₀ within the range of from <1 nM to >100 nM;
B. compound nos. 13-18, 25, 30-32, 38, 41-43, 55-58, 69-71, 77, 85, 92, 96, 98, 101, 113, 120, 121, 126, 128, 131, 137, 138, 141, 146-148, 165, 166, 173, 176, 181, 182, 184, 185, 193 and 194 had a PDE 5 IC₅₀ within the range of from >15 to 100 nM;
C. compound nos. 23, 24, 29, 33, 34, 39, 40, 93, 94, 108, 111, 112, 125, 136, 144, 160 and 161 had a PDE 5 IC₅₀ within the range of from >10 to 15 nM.
D. compound nos. 21, 22, 28, 36, 37, 59, 66, 68, 78, 79, 89, 95, 99, 110, 115, 132, 159, 171, 172, 175, 180, 183, 190 and 199 had a PDE 5 IC₅₀ within the range of from >5 to 10 nM; and, E. compound nos. 60-65, 67, 103-107, 114, 116-119, 122-124, 142, 168-170, 177, 178, 179, 186-188, 191, 197 and 198 had a PDE 5 IC₅₀ within the range of up to 5 nM.

In addition, another type of measurement that can be made is the ratio of PDE 5I IC₅₀/ PDE 5 IC₅₀ (identified as “PDE 5I/ PDE 5”), which is an indicator of enzyme selectivity—the higher the ratio, the more selective is the compound to inhibiting PDE 5 enzyme relative to PDE 5I enzyme. Measurements on the compounds (except for compound nos. 189, 192, 195 and 196) in Table II gave the following data (all numbers are modified by the word “about”):
F. compound nos. 1-188, 190, 191, 193, 194 and 197-199 had a PDE 5I/PDE 5 ratio of >0;
G. compound nos. 165 and 193 had a PDE 5I/PDE 5 ratio within the range of from >0 to 10;
H. compound nos.101, 108, 136, 141, 146, 148, 168, 173 and 194 had a PDE 5I/PDE 5 ratio within the range of from >10 to 25;
I. compound nos. 104, 125, 131-132, 137-138, 142, 144, 170, 175, 177, 185 and 199 had a PDE 5I/PDE 5 ratio within the range of from >25 to 50;
J. compound nos.103, 110, 111, 117, 159, 166, 182 and 187 had a PDE 5I/PDE 5 ratio within the range of from >50 to 75;
K. compound nos. 105, 106, 147 and 171 had a PDE 5I/PDE 5 ratio within the range of from >75 to 100;
L. compound nos.112, 113, 123, 124, 126, 169, 172 and 184 had a PDE 5I/PDE 5 ratio within the range of from >100 to 140; and
M. compound nos.107, 114-116, 118-122, 128, 160-161, 176, 178-181, 183, 186, 188, 190, 191, 197 and 198 had a PDE 5I/PDE 5 ratio of from >140.

Preferred compounds include those found in either of classes E and/or M: compound nos. 60-65, 67, 103-107, 114-124, 128, 142, 160-161, 168-170, 176-181, 183, 186-188, 190, 191, 197 and 198. More preferred are compounds found in both Classes E and M: nos. 107, 114, 116, 118, 119, 122, 178, 186, 188, 191, 197 and 198.

Another preferred compound of the invention has the following chemical structure:

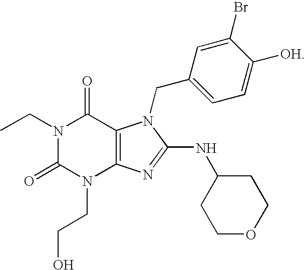

Specific and general procedures for producing representative compounds are disclosed in U.S. Pat. No. 6,821,978, which procedures are incorporated herein by reference. Obvious modifications to these procedures may be undertaken by one of ordinary skill in the art. Other compounds of the methods of the present invention may be produced using similar synthesis schemes.

This invention encompasses the use of any PDE 5 inhibitor for the treatment of BPH and/or LUTS. Thus, the use of sildenafil, tadalafil, vardenafil, or any other PDE 5 inhibitor is within the scope of the present invention.

### FORMULATIONS, DOSES AND COMBINATIONS

The compounds for use in the methods of the present invention may be administered to humans or other mammals by a variety of routes, including oral dosage forms and injections (intravenous, intramuscular, intraperitoneal, subcutaneous, and the like). The PDE 5 inhibitor compounds and their pharmaceutically-acceptable salts and neutral compositions may be formulated together with a pharmaceutically-acceptable excipients known in the art to form pharmaceutical compositions. Numerous dosage forms containing PDE 5 inhibitor compounds can be readily formulated by one skilled in the art, utilizing the suitable pharmaceutical excipients as defined below. For considerations of patient compliance, oral dosage forms are generally most preferred.

The pharmaceutically-acceptable carriers employed in conjunction with the compounds of the present invention are used at a concentration sufficient to provide a practical size to dosage relationship. The pharmaceutically-acceptable carriers, in total, may comprise from about 0.1 to 99.9% by weight of the pharmaceutical compositions of the invention, preferably, from about 20 to 80% by weight. Within the scope of the present invention are doses of about 2.5 mg. to about 250 mg., and preferably about 5 mg. to about 100 mg. Particularly preferred doses are 5, 10, 20, 25, 40 and 50 mg.

It may be desirable to initiate treatment at a relatively high dose, and upon improvement of a patient's condition, reduce the dose to a maintenance level. When the symptoms have been alleviated to the desired level, treatment should cease. Patients may, however, require intermittent treatment on a long-term basis upon any recurrence of disease symptoms. As a skilled artisan will appreciate, lower or higher doses than those recited above may be required.

The compounds of the present invention may be employed alone or in combination with other active agents, and it is understood that combinations with other active agents may be undertaken for treating benign prostatic hyperplasia or lower urinary tract symptoms while remaining within the scope of the invention. Additional agents known to a skilled formulator may be combined with the compounds for use in the methods of the invention to create a single dosage form. Alternatively, additional agents may be separately administered to a mammal as part of a multiple dosage form.

Examples of combinations within the scope of the invention include those with one or more of the following: finasteride, (α)1-AR blockers, prostanoids, α-adrenergic receptor, dopamine receptor agonists, melanocortin receptor agonists, endothelin receptor antagonists, endothelin converting enzyme inhibitors, angiotensin II receptor antagonists, angiotensin converting enzyme inhibitors, neutral metalloendopeptidase inhibitors, renin inhibitors, serotonin 5-HT_{2c} receptor agonists, nociceptin receptor agonists, rho kinase inhibitors, potassium channel modulators and inhibitors of multidrug resistance protein 5. Finasterade is the active ingredient in Proscar® (Merck). Examples of suitable (α)1-AR blockers include terazosin (brand name Hytrin®), prazosin (brand name Minizide®), doxazosin (brand name Cardura®), tamsulosin (brand name Flomax®) and alfuzosin (brand name Uroxatral®). Examples of ETA antagonists include bosentan, atrasentan, ambrisentan, darusentan, sitaxsentan, ABT-627, TBC-3711, Cl-1034, SPP-301, SB-234551, ZD-4054, BQ-123 and BE-18257B.

Further examples of combinations within the scope of the invention include those with one or more cardiovascular agents. Suitable cardiovascular agents are selected from the group consisting of thromboxane A2 biosynthesis inhibitors such as aspirin; thromboxane antagonists such as seratrodast, picotamide and ramatroban; adenosine diphosphate (ADP) inhibitors such as clopidogrel; cyclooxygenase inhibitors such as aspirin, meloxicam, rofecoxib and celecoxib; angiotensin antagonists such as valsartan, telmisartan, candesartran, irbesartran, losartan and eprosartan; endothelin antagonists such as tezosentan; phosphodiesterase inhibitors such as milrinoone and enoximone; angiotensin converting enzyme (ACE) inhibitors such as captopril, enalapril, enaliprilat, spirapril, quinapril, perindopril, ramipril, fosinopril, trandolapril, lisinopril, moexipril and benazapril; neutral endopeptidase inhibitors such as candoxatril and ecadotril; anticoagulants such as ximelagatran, fondaparin and enoxaparin; diuretics such as chlorothiazide, hydrochlorothiazide, ethacrynic acid, furosemide and amiloride; platelet aggregation inhibitors such as abciximab and eptifibatide; and GP IIb/IIIa antagonists.

When the invention comprises a co-administration of a PDE 5 inhibitor compound and one or more other therapeutically effective agents, the two or more active components may be co-administered simultaneously or sequentially, or a single pharmaceutical composition comprising a PDE 5 inhibitor compound and the other therapeutically effective agent(s) in a pharmaceutically acceptable carrier can be administered. The components of the combination can be administered individually or together in any conventional dosage form such as capsule, tablet, powder, cachet, suspension, solution, suppository, nasal spray, etc. The dosage of the other therapeutically active agent(s) can be determined from published material, and may range from 1 to 1000 mg per dose.

BPH/LUTS patients who are being treated with conventional methods can be treated adjunctively with PDE 5 inhibitors. Thus, PDE 5 inhibitors can be administered adjunctively with treatment by transurethral incision of the prostate (TUIP), transurethral resection of the prostate (TURP), open prostatectomy, laser prostatectomy, hyperthermia, prostatic stenting, or balloon dilation. The use of PDE 5 inhibitors may be most effective as adjunctive treatment to those procedures which do not actually remove or destroy the prostate, e.g., hyperthermia, prostatic stenting, or balloon dilation. The timing relative to the procedure and dosing of the PDE 5 treatment will be determined by the physician.

It is to be further understood that the above described methods and compositions apply to patients who suffer from BPH or LUTS, with or without suffering from erectile dysfunction (“ED”). Thus, for example, the inventive methods and compositions apply to patients who suffer from BPH, but not ED, and to patients who suffer from LUTS, but not ED.

The above description is not intended to detail all modifications and variations of the invention. It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims.

What is claimed is:

1. A method of treating benign prostatic hyperplasia or lower urinary tract symptoms comprising administering to a patient in need of such treatment an effective amount of at least one PDE 5 inhibitor compound, or an enantiomer, stereoisomer, rotomer, tautomer or a pharmaceutically acceptable salt thereof.

2. The method according to claim 1, wherein said at least one PDE 5 inhibitor compound is selected from the group consisting of Compound Nos. 10-199, as herein defined.

3. The method according to claim 1, wherein said at least one PDE 5 inhibitor compound is selected from the group consisting of Compound Nos. 60-65, 67, 103-107, 114-124, 128, 142, 160-161, 168-170, 176-181, 183, 186-188, 190, 191, 197 and 198, as herein defined.

4. The method according to claim 1, wherein said at least one PDE 5 inhibitor compound is selected from the group consisting of Compound Nos. 107, 114, 116, 118, 119, 122, 178, 186, 188, 191, 197 and 198, as herein defined.

5. The method according to claim 1, wherein said at least one PDE 5 inhibitor compound is selected from the group consisting of sildenafil, tadalafil, and vardenafil.

6. The method according to claim 1, wherein said at least one PDE 5 inhibitor compound is selected from the group consisting of:

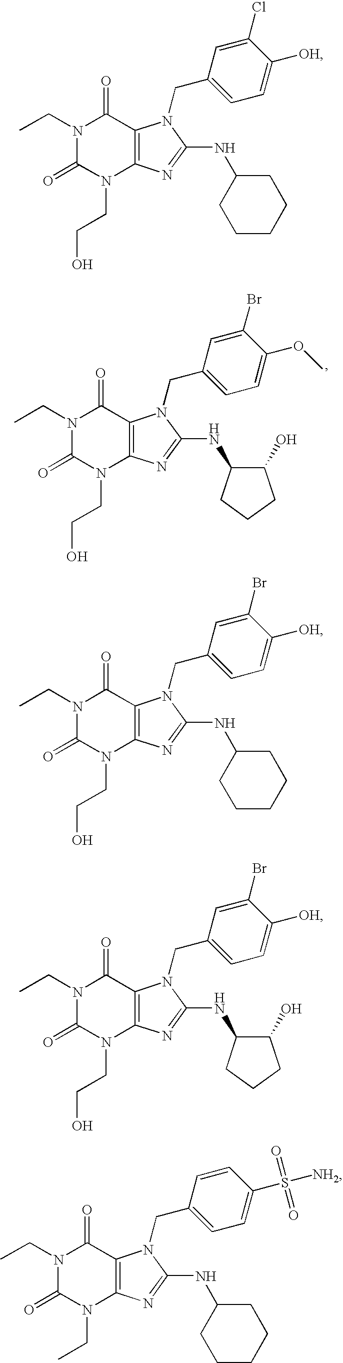

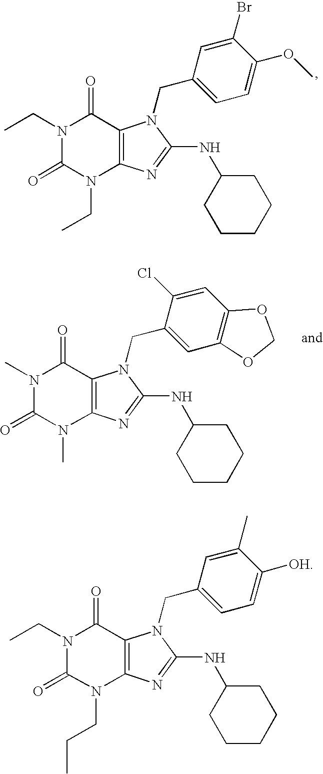

7. The method according to claim 1, wherein said at least one PDE 5 inhibitor compound is a compound of Formula (I), an enantiomer, stereoisomer, rotomer, tautomer or a pharmaceutically acceptable salt thereof: 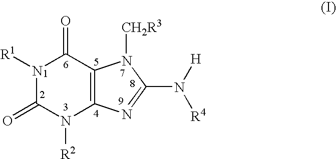
wherein:
(d) R¹ and R² are, independently of one another, each a C₁₋₁₅ alkyl group, branched or straight chain, unsubstituted or substituted with one or more substituents, a C₂₋₁₅ alkenyl group, branched or straight chain, unsubstituted or substituted with one or more substituents, a C₂₋₁₅ alkynyl group, branched or straight chain, unsubstituted or substituted with one or more substituents, or one of R¹ and R² is a hydrogen atom and the other one of R¹ and R² is defined the same as above;
(e) R³ is an aryl group, unsubstituted or substituted with one or more substituents, a heteroaryl group, unsubstituted or substituted with one or more substituents, or a heterocyclic group having 1 to 3 heteroatoms fused to a 5- or 6-membered aryl ring, unsubstituted or substituted with one or more substituents, with the proviso that R³ is not an aryl group substituted at its para position with a —Y-aryl group, where, Y is a carbon-carbon single bond, —C(O)—, —O—, —S—, —N(R²¹)—, —C(O)N(R²²)—, —N(R²²)C(O)—, —OCH₂—, —CH₂O—, —SCH₂—, —CH₂S—, —N(H)C(R²³)(R²⁴)—, —N(R²³)S(O₂)—, —S(O₂)N(R²³)—, —(R²³)(R²⁴)N(H)—, —CH═CH—, —CF═CF—, —CH═CF—, —CF═CH—, —CH₂CH₂—, —CF₂CF₂—, 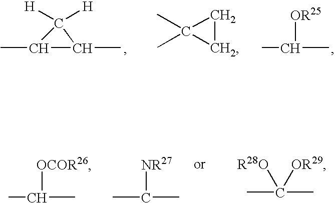
where,
R²¹ is a hydrogen atom or a —CO(C₁₋₄ alkyl), C₁₋₆ alkyl, allyl, C₃₋₆ cycloalkyl, phenyl or benzyl group;
R²² is a hydrogen atom or a C₁₋₆ alkyl group;
R²³ is a hydrogen atom or a C₁₋₅ alkyl, aryl or —CH₂-aryl group;
R²⁴ is a hydrogen atom or a C₁₋₄ alkyl group;
R²⁵ is a hydrogen atom or a C₁₋₈ alkyl, C₁₋₈ perfluoroalkyl, C₃₋₆ cycloalkyl, phenyl or benzyl group;
R²⁶ is a hydrogen atom or a C₁₋₆ alkyl, C₃₋₆ cycloalkyl, phenyl or benzyl group;
R²⁷ is —NR²³R²⁴, —OR²⁴, —NHCONH₂, —NHCSNH₂, 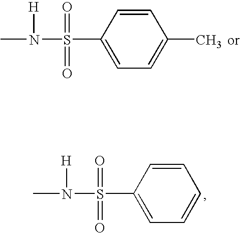
and
R²⁸ and R²⁹ are, independently of one another, each a C₁₋₄ alkyl group or, taken together with each other, a —(CH₂)q group, where q is 2 or 3; and
(f) R⁴ is a C₃₋₁₅ cycloalkyl group, unsubstituted or substituted with one or more substituents, or a C₃₋₁₅ cycloalkenyl group, unsubstituted or substituted with one or more substituents;
wherein, the one or more substituents for all the groups are chemically-compatible and are, independently of one another, each an: alkyl, cycloalkyl, alkenyl, cycloalkenyl, alkynyl, arylalkyl, alkylaryl, aryl, heteroaryl, heterocycloalkyl, hydroxyalkyl, arylalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, alkoxy, hydroxy, halo, nitro, oximino, —COOR⁵⁰, —COR⁵⁰, —SO₀₋₂R⁵⁰, —SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, —CON(R⁵⁰R⁵¹), —OCOR⁵⁰, —OCON(R⁵⁰R⁵¹), —N(R⁵²)CO (R⁵⁰), —N(R⁵²)COOR⁵⁰ or —N(R⁵²)CON(R⁵⁰R⁵¹) group, where:
R⁵⁰, R⁵¹ and R⁵² are, independently of one another, each a hydrogen atom or a branched or straight-chain, optionally substituted, C₁₋₆ alkyl, C₃₋₆ cycloalkyl, C₄₋₆ heterocycloalkyl, heteroaryl or aryl group, or R⁵⁰ and R⁵¹ are joined together to form a carbocyclic or heterocyclic ring system, or R⁵⁰, R⁵¹ and R⁵² are, independently of one another, each: 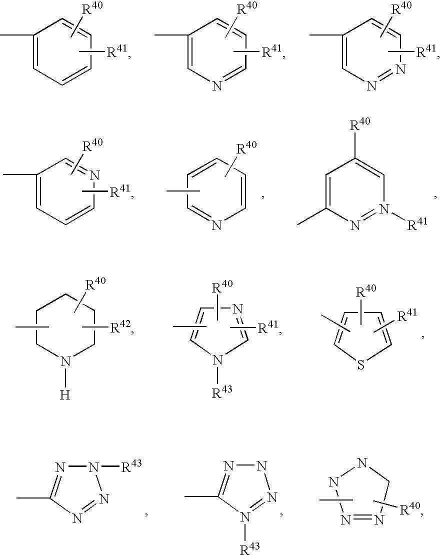
where,
R⁴⁰ and R⁴¹ are, independently of one another, each a hydrogen atom or a branched or straight-chain, optionally substituted, alkyl, heteroaryl, arylalkyl, arylalkoxy, heteroarylalkyl, heteroarylalkoxy, aminoalkyl, haloalkyl, mono-, di- or trihaloalkyl, mono-, di- or trihaloalkoxy, nitro, cyano, alkoxy, hydroxy, amino, phosphino, phosphate, alkylamino, dialkylamino, formyl, alkylthio, trialkylsilyl, alkylsulfonyl, arylsulfonyl, alkylsulfinyl, aminoalkyl, alkylaminoalkyl, dialkylaminoalkyl, hydroxyalkyl, morpholino, thioalkyl, alkylthioalkyl, carboxyalkyl, oximino, —COOR⁵⁰, —COR⁵⁰, —SO₀₋₂R⁵⁰, —SO₂NR⁵⁰R⁵¹, —NR⁵²SO₂R⁵⁰, —CON(R⁵⁰R⁵¹), —OCON(R⁵⁰R⁵¹), —N(R⁵²)CO(R⁵⁰), —N(R⁵²)COOR⁵⁰, —N(R⁵²)CON(R⁵⁰R⁵¹) or —OCONR⁵⁰ group, where, R⁵⁰, R⁵¹ and R⁵² are defined the same as above;
R⁴² is a hydrogen atom or a branched or straight-chain, optionally substituted, alkyl, alkenyl, arylalkyl or acyl group; and
R⁴³ is a hydrogen atom or a branched or straight-chain, optionally substituted, alkyl or aryl group;
wherein, the optional substituents are defined the same as above for the one or more substituents.

8. The method of claim 1 further comprising administering to the patient an effective amount of at least one active agent selected from the group consisting of finasteride, (α)1-AR blockers, prostanoids, α-adrenergic receptor, dopamine receptor agonists, melanocortin receptor agonists, endothelin receptor antagonists, endothelin converting enzyme inhibitors, angiotensin II receptor antagonists, angiotensin converting enzyme inhibitors, neutralmetalloendopeptidase inhibitors, renin inhibitors, serotonin 5-HT_{2c} receptor agonists, nociceptin receptor agonists, rho kinase inhibitors, potassium channel modulators and inhibitors of multidrug resistance protein 5.

9. The method of claim 8 wherein said PDE 5 inhibitor compound is:

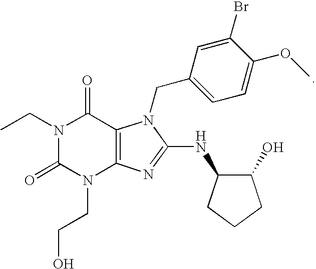

10. The method of claim 1 further comprising administering to the patient an effective amount of at least one (α)1-AR blocker selected from the group consisting of terazosin, prazosin, doxazosin, tamsulosin and alfuzosin.

11. The method of claim 10 wherein said PDE 5 inhibitor compound is:

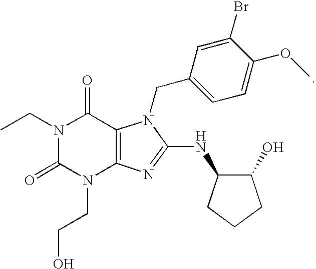

12. The method of claim 1 further comprising administering to the patient an effective amount of at least one ET_{A} antagonist selected from the group consisting of bosentan, atrasentan, ambrisentan, darusentan, sitaxsentan, ABT-627, TBC-3711, CI-1034, SPP-301, SB-234551, ZD-4054, BQ-123 and BE-18257B.

13. The method of claim 12 wherein said PDE 5 inhibitor compound is:

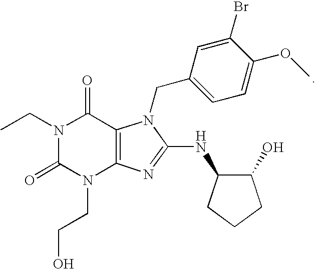

14. The method according to claim 1, further comprising administering to said patient at least one cardiovascular agent selected from the group consisting of thromboxane A2 biosynthesis, thromboxane antagonists, adenosine diphosphate (ADP) inhibitors, cyclooxygenase inhibitors, angiotensin antagonists, and ET_{A} antagonists.

15. The method of claim 14 wherein said PDE 5 inhibitor compound is:

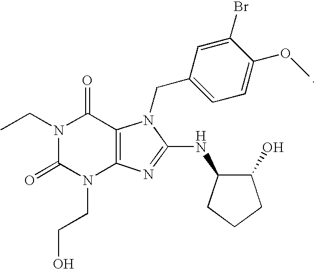

16. The method according to claim 1, further comprising treating said patient with a procedure selected from the group consisting of prostatic hyperthermia, prostatic stenting, and balloon dilation.

17. The method of claim 16 wherein said PDE 5 inhibitor compound is:

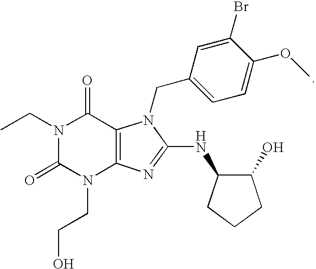

18. The method of claim 1 wherein said patients do not suffer from erectile dysfunction prior to said treating.

19. A method of treating benign prostatic hyperplasia or lower urinary tract symptoms comprising administering to a patient in need of such treatment an effective amount of at least one PDE 5 inhibitor compound, wherein said compound is:

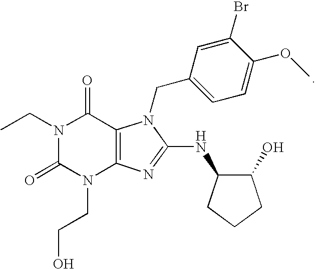

20. A pharmaceutical composition for treating benign prostatic hyperplasia or lower urinary tract symptoms, said composition comprising an effective amount of at least one PDE 5 inhibitor compound and a pharmaceutically acceptable excipient.

21. The composition of claim 20 wherein said at least one PDE 5 inhibitor compound is: [chemical structure]
